(19) United States
(12) Patent Application Publication
(10) Pub. No.: US 2001/0044569 A1
(43) Pub. Date: Nov. 22, 2001

(54) Penile enlargement device

(76) Inventor: Monshi Naghar, London (GB)

Correspondence Address: PERKINS COIE LLP, PATENT-SEA, P.O. BOX 1247, SEATTLE, WA, 98111-1247, US

(21) Appl. No.: 09/734,320

(22) Filed: Dec. 1, 2000

Related U.S. Application Data
(63) Continuation of application No. PCT/GB99/01702, filed on May 28, 1999.

(30) Foreign Application Priority Data
Jun. 3, 1998 (GB) 9811946.4

(51) Int. Cl. A61F 5/00

(52) U.S. Cl. 600/38

(57) ABSTRACT

Penile enlargement device comprising a rigid tube having a closed end and a restricted opening for receiving a penis at the other end. A selectively closable opening is provided on the tube. In use, as a penis is inserted through the restricted opening, air is expelled through the selectively closable opening. Relative movement between the tube and the penis increases the vacuum within the tube enhancing the flow of blood into the penis, and thereby increasing the stiffness of the penis.

### CROSS-REFERENCE

The present application is a continuation of prior International Application No. PCT/GB99/01702, filed May 28 1999 and published as International Publication No. WO 99/53872, which in turn claims priority to Brittish Application No. 9811946.4, filed Jun. 3, 1998. The entire disclosures of both of the prior applications, as well as Internatinal Publication No. WO 99/53872, are incorporated herein by reference.

### BACKGROUND OF THE INVENTION

This invention relates to a penile erection and enlargement device.

Known penile enlargement devices comprise a sleeve, one end of which is provided with a flexible gasket with a hole to accommodate a penis. The other end is closed and has a connection to a vacuum pump which may be a manually operated pump or may be a motor driven one. The flaccid penis is inserted through the gasket into the sleeve and then the air in the sleeve is removed by means of the pump. As a result the penis is encouraged to enlarge and blood entering the penis is trapped by the constriction of the flexible gasket.

There are problems with such a known device. In some cases the hole in the gasket may be too small for the penis to fit through, and in other cases the hole will be too large and therefore it will not be possible to obtain an acceptable air-tight seal. Also the sleeve must usually be of large diameter relative to the size of a penis to accommodate the flexible gasket and be of a sufficient length to accommodate various penis lengths. Therefore it may be necessary to remove a large volume of air from the device. A light duty rubber hand pump is usually used, and the vacuum which can be created within the sleeve is often at a rather modest level. This is why manufacturers recommend the use of an electrically operated vacuum pump. This is expensive and bulky however. Even where an electrically operated pump is used, the vacuum inside the sleeve will tend to pull the flexible gasket in which may allow air to seep into the tube before a satisfactory vacuum level is reached.

### SUMMARY OF THE PRESENT INVENTION

According to the present invention a penile enlargement device comprises a tube made of rigid material which is closed at one end and includes a first opening of constricted area at the other end, a second opening and a means for selectively closing the second opening.

With such an arrangement, when the well lubricated flaccid penis is slid into the tube through the first opening, it will form a good seal around the penis or the flesh around the end of the penis. The constricted diameter portion of the tube will not have an uncomfortable feeling like a tight and strong rubber gasket. With the second opening opened, the insertion action will cause air in the sleeve to be expelled through the second opening. The second opening is then closed with the closure means. The penis is now trapped within the tube. By pulling on the tube, the tube will slide along the penis, partially withdrawing the penis from the tube. This will increase the vacuum within the tube, as the partially removed penis cannot be replaced by air as the penis seals the first opening and as the second opening is closed by the closure means. This increased vacuum will encourage blood to flow in a one way direction into the penis, so enhancing its size, and the firmness of its erection. The device according to the invention will dramatically expand the veins and the spongy material in the penis, allowing an increased volume of blood in the penis. This will help people with impotence and also allow stretching to help men with a small penis. Further, the restricted opening of the tube will have the effect of restraining the outflow of blood from the penis so keeping it erect. To remove the device, the second opening is opened to allow air into the tube and to release the vacuum within the tube.

With such an arrangement, there is no need to provide a vacuum pump to remove air from the tube as the air is displaced by the insertion of the penis into the tube. Therefore the level of vacuum within the tube is dependent solely on the amount of relative movement between the tube and the penis. This greatly simplifies the device, increasing its reliability and effectiveness.

The diameter of the tube over the major part of its length will be of a size slightly larger than the erect penis so that there is very little remaining air space within the tube, but allows for some expansion of the penis within the tube. In this way, when the tube is pulled forward, a powerful vacuum will be created in the tube, preventing the erect penis from deflating, whilst giving some space for the penis to expand. It is desirable that the diameter of this major part of the sleeve be in the range of from 25 mm to 80 mm and preferably in the range of 30 mm to 60 mm. The thickness of the wall of the tube is preferably between 1 and 10 mm, and more preferably is around 2.5 mm.

The constricted portion of the device preferably extends over a length of less than 40 mm, preferably of 1 mm to 20 mm, and more preferably from about 2 mm to 10 mm. It may be of uniform diameter, but is desirably shaped so as to include a portion of progressively narrowing diameter which converges from the diameter of the main portion of the tube. The constricted portion is preferably in the range of from 25 mm to 70 mm, and more preferably between 30 mm and 50 mm in diameter which will engage with the penis to give the required air-tight seal. Preferably the constricted portion leads into a slightly enlarged mouth through which the penis is inserted. This mouth is preferably of outwardly fluted shape and includes a rounded outwardly extending integral flange to assist in guiding the penis into the sleeve and avoiding sharp edges.

The overall length of the device will generally range from 90 mm to 300 mm and is preferably less than 250 mm, more preferably less than 180 mm. The overall unconstricted length of the tube is most preferably in the range of 90 mm to 250 mm. Devices in a range of lengths and other sizes can be provided to suit a range of users.

The sleeve is preferably made of transparent material so that the effect on the penis can readily be monitored. Also it is then possible to check whether the inside of the tube is dirty and needs to be cleaned. The tube could therefore be made of glass or of plastics material. The tube may be molded. Alternatively the device could be made of a metal such as aluminium for longer life.

The tube may be cylindrical, although it may be advantageous to include internal ribs to give additional restrictions, and may be bent or otherwise shaped. The tube may include sloping walls.

Advantageously, the outer surface of the tube is shaped to facilitate gripping and pulling of the tube, for example by including projections or recesses formed integrally with the tube or a projection fixed to the tube.

The second opening is preferably provided at or near the closed end of the tube opposite the end including the first opening. This ensures that the penis does not block the opening and prevent air passing through the opening. The second opening may be a hole which is closable by any suitable means, such as a stopper or cap, which allows the hole to be opened again. In this case it is preferred that the hole is provided through a threaded flange and the cap is screwable onto the flange to seal the hole. Alternatively, the second opening and closure means may be in the form of a one-way valve which allows air to pass through the valve from within the tube, but which does not allow air to enter the tube until the valve is released.

### BRIEF DESCRIPTION OF THE DRAWINGS

An example of a penile erection device according to the invention will now be described with reference to the accompanying drawings, which shows a side view, partially cross-sectioned, of a penile erection device.

### DETAILED DESCRIPTION OF PREFERRED EMBODIMENTS

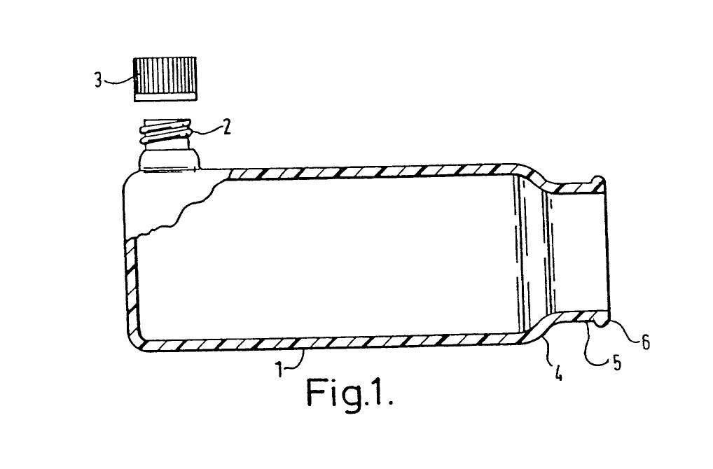

As shown in FIG. 1, in one example the penile erection device comprises a tube 1 of rigid transparent plastics material having a substantially constant diameter along its length. The left hand end of the tube 1 as shown in the drawing is closed, but includes an outwardly projecting outlet 2. The outlet 2 has an outer threaded portion and a through hole for communication between the inside of the tube 1 and the atmosphere. A cap 3 is provided which includes an internal threaded portion corresponding to the external threaded portion of the outlet 2. This allows the cap 3 to be screwed onto the outlet 2 to seal the outlet 2, and to be removed to allow communication through the outlet 2.

The right hand end of the tube 1, as viewed in the drawing, has an integral narrowing portion 4 which merges smoothly into the main body of the tube 1. This is in turn joined to a constricted or sealing portion 5 of substantially constant but restricted diameter designed to seal against the penis. Finally an open mouthed portion 6 of fluted or slightly flared shape is integrally joined to the portion 5 and includes an outwardly projecting rounded thickened flange. In this way there are no sharp edges.

In operation, a well lubricated flaccid penis is slid into the tube 1 through the portion 5. As the penis is inserted into the tube 1, air within the sleeve is expelled through the outlet 2. The cap 3 is then closed to seal the opening 2 to prevent air entering the tube 1. Gentle movement of the tube 1 with respect to the penis will cause the penis to partially slide from the tube 1 and this will accentuate the vacuum in the tube. This encourages the penis to expand as blood flows into the penis and accordingly causes an erection. The slightest pull will encourage blood to flow inside and the sliding action will cause a harder erection. By unscrewing the cap 3 to open the outlet 2, the enlarged penis will be able to expel additional air through the outlet 2 which can then again be closed by the cap 3, thereby maintaining a suitable vacuum within the tube 1. The process may then be repeated.

As an alternative to the screw-on cap, the cap may be press-fitted to seal the outlet.

As an alternative to the stopper, a one way valve can be used to close selectively the opening. In this case, the one way valve, which may include a valve stopper biased towards a valve seat, is arranged to allow air to be expelled from the tube, but prevents air entering the tube.

1. A penile enlargement device comprising a tube of rigid material which is closed at one end and includes a first opening of constricted area at the other end for receiving a penis, and a selectively closeable second opening, characterised in that, in use, the second opening may be opened as a penis is inserted into the tube through the first opening, thereby expelling air from the tube, and may be closed to seal the tube, such that the tube may be slid along the penis thereby reducing the pressure in the tube to cause blood to flow into the penis.

2. A device according to claim 1 in which the diameter of the major part of the tube is in the range of from 25 mm to 80 mm, and preferably in the range of 30 mm to 60 mm.

3. A device according to claim 1 or 2 in which the constricted diameter portion of the device extends over a length of 1 mm to 20 mm, and preferably from 2 mm to 10 mm.

4. A device according to claim 1 in which the constricted diameter portion is shaped so as to include a portion of progressively narrowing diameter which converges from the diameter of the main part of the tube.

5. A device according to claim 4 in which the narrowing portion leads into a constricted portion of approximately constant diameter, preferably in the range of from 25 mm to 70 mm.

6. A device according to claim 1 in which the constricted portion leads into an enlarged mouth through which the penis is inserted.

7. A device according to claim 1 in which the overall length of the device ranges from 80 mm to 300 mm.

8. A device according to claim 7 in which the overall length of the device is not more than 180 mm.

9. A device according to claim 1 in which the overall unconstricted length of the tube is in the range of 90 mm to 250 mm.

10. A device according to claim 1 in which the tube is made of transparent material.

11. A device according to claim 1 in which the tube is made of glass or of plastics material.

12. A device according claim 1 in which the tube includes internal ribs.

13. A device according claim 1 in which the outer surface of the tube is shaped to facilitate gripping and pulling of the tube.

14. A device according to claim 1 in which the second opening is provided at or near the closed end of the tube opposite the end including the first opening.

15. A device according to claim 1 in which the second opening is a hole which is closable by a stopper or cap.

16. A device according to claim 1 in which the second opening includes a one-way valve which allows air to pass outwardly through the valve from within the tube, but which does not allow air to enter the tube until the valve is released.

17. A penile enlargement device comprising a tube of rigid material which is closed at one end, the tube including a first opening of constricted area at an opposite end for receiving a penis, a second opening, and a stopper or cap associated with the second opening such that the second opening may be opened as a penis is inserted into the tube through the first opening, thereby expelling air from the tube, yet may be closed with the stopper or cap to seal the tube such that the tube may be slid along the penis, thereby reducing the pressure in the tube to cause blood to flow into the penis.

18. A method of enlarging a penis using a tube of rigid material which is closed at one end, the tube including a first opening of constricted area at an opposite end for receiving a penis and a selectively closable second opening, comprising:
with the second opening open, inserting the penis into the tube through the first opening, thereby expelling air from the tube through the second opening;
closing the second opening to seal the tube; and
sliding the tube along the penis, thereby reducing pressure in the tube to cause blood to flow into the penis.
